(19) United States
(12) Patent Application Publication
(10) Pub. No.: US 2024/0181221 A1
(43) Pub. Date: Jun. 6, 2024

(54) SAFETY CANNULA ASSEMBLY

(71) Applicant: Sarstedt AG & Co, KG, Nuembrecht (DE)

(72) Inventor: Viktor HAUPT, Nuembrecht (DE)

(21) Appl. No.: 18/532,932

(22) Filed: Dec. 7, 2023

Related U.S. Application Data
(63) Continuation of application No. PCT/EP22/65975, filed on Jun. 13, 2022.

(30) Foreign Application Priority Data
Jun. 15, 2021 (DE) DE102021115548.7

(51) Int. Cl. A61M 25/06; A61M 25/00

(52) U.S. Cl. CPC A61M 25/0631; A61M 2025/006

(57) ABSTRACT

A safety cannula assembly, comprising a cannula configured to puncture human or animal tissue, wherein a distal end section of the cannula includes a tip; a sliding body including a distal end section connected with the cannula and a proximal end section connected with a flexible tube, wherein a flow connection between the tip of the cannula and a proximal end of the flexible tube runs through the sliding body; a main body including an interior space with the sliding body movably supported in the interior space; an actuation element arranged between the main body and the sliding body and configured to displace the sliding body from an operating position where the tip of the cannula is located outside the main body to a safety position where the tip of the cannula is arranged inside the main body.

### RELATED APPLICATIONS

This application is a continuation of International patent application PCT/EP2022/065975 filed on Jun. 13, 2022 claiming priority from German Patent application DE 10 2021 115 548.7 filed on Jun. 16, 2021, both of which are incorporated in their entirely by this reference.

### FIELD OF THE INVENTION

The invention relates to a safety cannula assembly, for puncturing human or animal tissue. The instant invention primarily relates to safety cannula assemblies with active needle protection.

An actuation element according to the instant invention is an element configured to impart a required force upon a sliding body functioning as a drive to move the sliding body from an operating position into the safety position. A drive element and an associated energy storage device can be separate from each other. However, the two functions can be combined in one element, in particular configured as spring element that either store mechanical energy in the form of a coil spring and/or pressure energy like e.g. in a preloaded gas storage device.

The main body according to the invention is a housing configured to house at least a portion of the sliding body. The main body includes an interior configured to receive the sliding body and in particular the cannula tip in the safety position. Walls of the main body do not have to be completely closed in this context but can also be configured as a cage and can in particular have cut outs, interruptions, slots, or other perforations.

A sliding body according to this application is an element which receives the cannula at a distal end and which is connected with a tube at a proximal end. The sliding body is typically also designated as cannula holder or needle carrier.

### BACKGROUND OF THE INVENTION

A needle arrangement is known from EP 3 466 464 A1 including a sliding element with a proximal thickness increase. The cross section in the thickened portion is configured oval, a maximum diameter is greater than a maximum diameter of the main body which is also oval at its proximal end. The main body is also significantly thicker at its proximal end compared to an adjoining section that extends in the distal direction. The trigger mechanism is arranged in this thickened section at the sliding body. Pressure upon two keys having checkered surfaces and arranged diametrically opposed to a longitudinal axis of the sliding body moves two spring elastic tongues inward which puts locking elements out of engagement that engage recesses in the thickened portion of the base element. After the locking elements are moved from a locking position into a release position by radial pressure from an outside, a pullback movement of the sliding body and thus by a pullback movement of cannula can be performed in the proximal direction. The cannula in particular, the tip is thus displaced into the interior of the main body. It is a disadvantage of this known cannula arrangement that the sliding body and thus the cannula has to be manipulated (this means moved) by the person using the cannula arrangement when performing the pullback movement or before performing the pullback movement. This may cause unintentional movements or forces in the area of the cannula tip that are uncomfortable or painful for the patient while cannula tip is still within the tissue of the patient.

A safety cannula arrangement that has a similar configuration is disclosed in EP 3 169 387 B1. The proximal ends of the sliding body and of the main body are thickened. Contrary to EP 3 466 464 A1 discussed supra, the trigger mechanism of EP 3 169 387 B1 is arranged at the proximal thickened end of the main body. Checkered pressure surfaces are in turn arranged at keys arranged diametrically opposed to each other and forming spring elastic tongues and having hook shaped locking elements at their proximal ends. Radial pressure upon the keys in inward direction also displaces the locking elements radially inward and moves them out of engagement at retaining surfaces that are arranged radially further outward at an edge of recesses in the thickened end section of the sliding body. A spring element arranged between the sliding body and the main body causes the sliding body including the cannula arranged thereon to perform the pullback movement after triggering. It is advantageous in this context that the manipulation is performed upon the stationary main body, whereas the pullback movement of the sliding body cannot be interfered with by the person using the cannula arrangement and touching the sliding body. By the same token it is disadvantageous that the proximal end of the sliding body has a greatly enlarged diameter compared to the remaining portion of the sliding body which can impair the pullback movement due to skin contact with the patient and induced friction forces. Also, this can lead to an uncontrolled pull back movement and thus cause the patient pain.

WO 2016-007438 A1 discloses a safety cannula arrangement where the trigger mechanism is configured as a tongue that is spring elastic in the radial direction. The tongue protrudes from and through an accordingly configured recess in the main body with a key or push-button section of the tongue and is thus actuatable from an outside by a finger of a person using the safety cannula arrangement. Applying a radially inward oriented force upon the key section a locking element arranged at the tongue and configured as a shoulder is caused to disengage from the wall of the recess in the main body described supra. Thus, the tongue penetrates an accordingly configured pocket in the sliding body so that the tongue can penetrate in a next step axially into an inner free cross section of the main body. An actuator configured as a coil spring thus causes a pull-back movement of the sliding body including the cannula arranged thereon in a proximal direction. It is a disadvantage of this prior art arrangement that the configuration and fabrication of the sliding body with the elongated tongue of the trigger mechanism is complicated. An accidental triggering of the needle protection is possible due to the light and externally accessible trigger wherein this triggering can already be caused by loads caused by transporting. The force introduced by the triggering is not symmetrical and transferred to the needle tip. This inevitably irritates the patient.

Another safety cannula with a proximally thickened sliding body and a trigger mechanism arranged thereon and a thickened end section of the main body is known from WO 2015/058402 A1. The disadvantages are essentially the same as the disadvantages of the device EP 3 466 464 A1.

Additionally, WO 2009/021263 A1 discloses a safety cannula arrangement where the trigger mechanism is arranged at the main body having a respective spring elastic tongue in a distal end section on diametrically opposed sides wherein the tongue is displaceable inward by a radial pressure from two fingers of the person operating the device. The tongues of the main body pull along two spring elastic tongues of the sliding body that are oriented approximately parallel to the tongues of the main body during the radially inward displacement. This moves the tongues of the sliding body radially inward as well.

The springs of the sliding body are configured with face side locking elements at rear ends of the springs which contact walls of a cut out in the main body. This contact is broken by the displacement of the spring elastic tongues of the sliding body in the inward direction which releases the sliding body so that the sliding body is displaced in the proximal direction by a force of a coil spring and moves the cannula into the safety position. The configuration of the main body as well as the configuration of the sliding body are complicated in this prior art device due to its special type of trigger mechanism. Due to the thickness increase of the sliding body in a proximal section and a radial step formed at a transition portion to the flexible tube there is a risk that the pull back movement of the sliding body and the tube is impeded by the skin contact at the patient.

Additionally, U.S. Pat. No. 5,575,777 discloses a device for placing a catheter where energy absorbing components e.g., configured as a viscous material are arranged in an interior of the base body of the device in order to reduce a velocity of the needle carrier during the pull back movement. Thus, a pull-back mechanism is associated with the needle carrier and includes a trigger mechanism and a locking mechanism. The locking mechanism includes a combined trigger and locking element which is designated as a slider or trigger and is formed by a slide that is displaceable radially, this means in a plane perpendicular to the longitudinal axis of the main body or of the cannula. The slide includes a key hole shaped recess in a center portion. The slide engages the cannula carrier in the locking position in a portion of a circumferential groove at a distal end of the cannular carrier. A front end of the cannula carrier that is thickened compared to the circumferential groove prevents that the cannula carrier preloaded by the spring tension is moved from the operating position into the safety position. The walls of the circumferential groove are supported at wall sections of the slide in this condition which define the key hole at both sides in an approximately rectangular narrow portion of the key hole.

The slide is now manually moved transversal to a longitudinal axis of the needle by a pressure upon an outer trigger member so that the thickened front end of the slide moves into a larger approximately circular section of the key hole, so that the front end of the cannula holder disengages from the slide so that the pull back movement is triggered.

The known device configured as a placement aid for a catheter is not configured as a blood drawing device that is used for drawing a bodily fluid, in particular blood, from a patient. The cannula itself is not configured for a flow through in the sense of a liquid draw or infusion. The proximal end of the cannula rather leads into a chamber that is closed by a hydrophilic filter at a distal end in order to facilitate a flow out of air but not an exit of blood when the cannula is punctured into the skin of the patient. Reconfiguration of this device for catheter placement into a safety cannula arrangement for blood drawing is therefore not possible.

Additional safety cannulas with a comparative configuration are known from U.S. Pat. No. 6,547,762 and EP 2 638 924 A1.

EP 1 479 408 A1 describes a needle safety arrangement where the trigger mechanism is arranged at the needle carrier and causes a disadvantageous thickness increase at the possible end of the needle carrier as described supra. Besides the locking elements at the spring elastic tongues of the trigger mechanism arranged at the needle carrier the trigger mechanism is arranged in its entirety adjacent to the main body viewed in the axial direction, this means in a section between the tube connection and the proximal end of the main body. It is another disadvantage of this needle safety arrangement that fingers of the person operating the trigger mechanism have to follow the pullback movement of the sliding body synchronously or that the fingers have to be removed from the trigger mechanism immediately after activating the trigger mechanism in order to facilitate a free backward movement of the sliding body that is not impeded by the fingers of the operating person.

### BRIEF SUMMARY OF THE INVENTION

Improving upon the prior art described supra it is an object of the invention to propose a safety cannula arrangement that is easy to operate, has little engineering complexity and production complexity and minimizes pain or trauma in the patient during or after triggering the pullback mechanism for the sliding body.

### Solution

Improving upon a safety cannula arrangement of the type described supra, the object is achieved by A safety cannula assembly, comprising: a cannula configured to puncture human or animal tissue, wherein a distal end section of the cannula includes a tip; a sliding body including a distal end section connected with the cannula and a proximal end section connected with a flexible tube, wherein a flow connection between the tip of the cannula and a proximal end of the flexible tube runs through the sliding body; a main body including an interior space with the sliding body movably supported in the interior space; an actuation element arranged between the main body and the sliding body and configured to displace the sliding body from an operating position where the tip of the cannula is located outside the main body to a safety position where the tip of the cannula is arranged inside the main body; and a trigger mechanism arranged at the main body and configured to trigger a displacement of the sliding body from the operating position to the safety position, wherein the trigger mechanism includes at least one trigger member loadable with a compressive force by a finger of a person using the safety cannula assembly and provided with at least one locking element displaceable from a locking position where the locking element engages the sliding body and locks the sliding body in the operating position, to a trigger position, where the locking element does not engage the sliding body so that the sliding body moves into the safety position, wherein a distance of the at least one locking element from a longitudinal axis of the sliding body is increased when the at least one locking element is displaced from the locking position to the trigger position, wherein the at least one trigger member and the associated at least one locking element are located on different or on opposite sides of the longitudinal axis of the sliding body.

The safety cannula arrangement is advantageously configured as a blood drawing device.

The arrangement of trigger member and locking element on opposite sides of the longitudinal axis of the sliding body facilitates transposing a radially inward pressure upon the trigger member, this means oriented towards the longitudinal axis, and a radially inward movement of the trigger member caused thereby into a movement of the associated locking element into a direction oriented radially away from the longitudinal axis or having at least a movement component in this direction in a simple manner. An “associated locking element” is any coupling between a trigger member and a cooperating locking element, in particular a mechanical forced coupling, in particular a connection by pressure forces and/or shear forces and/or torques transferred by an intermediary member which can be integrally connected with the trigger member and/or the locking member.

The movement of the locking element in the radially outward direction caused by a radially inward movement of the trigger member is particularly advantageous since the radially outward oriented movement of the locking element facilitates an increase and/or shape change of a free cross section in an interior in the main body in simple manner. This cross section increases or shape change facilitates establishing a necessary clear space for the pullback movement of the sliding body. This means that the locking element is moved from a position where it engages the sliding body and where it is arranged closer to the longitudinal axis of the sliding body into a trigger position or release position where it is arranged radially further away from the longitudinal axis of the sliding body. The trigger principle according the invention thus transposes a radially inward movement of the trigger element into a radially outward movement of the associated locking element which implements a simple configuration of the locking mechanism that requires little installation space in the radial direction. In particular the sliding body can be kept completely clear from the trigger mechanism and the trigger members which facilitates a slender and simple configuration. Thus, the manipulation of the safety cannula arrangement according the invention is only performed at the main body and not at the sliding body so that the sliding body can slide backward freely and in particular without contact with the fingers of the person using the safety cannula arrangement after activating the trigger mechanism. The slender shape of the sliding body without large outward protruding components of a trigger mechanism facilitates an unimpeded pullback movement and prevents in particular unintentional contact of the skin of the patient that would slow down the pullback movement of the sliding body.

Advantageously the at least one trigger member and the associated at least one locking element are arranged on different sides of a plane that runs parallel to a longitudinal axis of the cannula and also perpendicular to a plane that is defined by a ground surface of the cannula tip.

Advantageously the safety cannula arrangement includes at least one grip lobe at the main body. An arrangement of two grip lobes on two opposite sides of the main body is particularly advantageous.

According to an advantageous embodiment of the invention, a respective trigger member and a respective locking element is arranged at a cross section oriented perpendicular to the longitudinal axis of the sliding body at diametrically opposite sides of the longitudinal axis. Thus, two pairs respectively made from a trigger member and an associated locking element are arranged on opposite sides cooperate with each other. This symmetry renders the configuration of the safety cannula arrangement according to the invention particularly simple and makes operating it safe and intuitive. It is appreciated that four pairs of trigger member and associated locking element can also be provided instead of two pairs. As a matter of principle an arrangement of three pairs, thus respectively offset from each other by 120 degrees in the circumferential direction can be used though this is not a preferred solution.

A mechanical forced coupling between trigger member and locking member can be achieved in a particularly simple manner when at least one trigger member and the associated locking element form a U-Shape or C-shape in combination in a cross section of the main body that runs orthogonal to the longitudinal axis of the sliding body. In this context it is particularly advantageous when the at least one trigger member is arranged at a first arm of the U or the C and the associated locking element is arranged at a second arm of the U or C.

According to an advantageous embodiment of the safety cannula arrangement according to the invention each trigger member is connected with the main body by at least one link element, where in the link element is deformable in an elastic and/or plastic manner relative to the main body when the trigger member is moved from the locking position into the trigger position. A link element facilitates a relative movement between the trigger member and the main body without requiring large forced application by the person using the safety cannula arrangement.

The link element is configured as a bar according to an advantageous embodiment. Advantageously plural link elements are configured as bars that run parallel to one another and are advantageously equidistant. This way a flat grid structure is provided which is also suitable in particular as a main body side wall and which also has the elasticity required for activating the trigger mechanism.

According to a particularly advantageous embodiment of the invention the at least one link element is connected with a proximal end at a proximal end section of the main body and provided with the locking element at a distal end. In this embodiment the force is transferred through the main body and advantageously through a rigid side wall that is sufficiently stable against bending so that the force is transferred the proximal end and forms a bearing for the link element at this location. A transversal force applied to the trigger member is thus transferred through the link element under elastic deformation to the bearing point of the link element at the proximal end and reacted from there through a side wall in a direction of the remaining main body. This way a sufficiently large distance of the at least one locking element can be achieved from the proximal end of the main body, wherein the at least one locking element is arranged at the distal end of the link element. A terminating portion for the flexible tube can be arranged within the main body in the trigger section in this configuration.

Furthermore, plural link elements advantageously configured as bars are connected at their distal ends by a compression rod running perpendicular to the bar, wherein the compression rod connects a trigger member with the associated locking element. The trigger member, the intermediate compression rod and the locking element connected thereto can form a U-shape or a C-shape in a plane orthogonal to the longitudinal axis of the main body.

When the trigger member includes a trigger surface that is arranged at an angle of 90 degrees relative to a plane in which the at least one link element is arranged and/or a compression rod connecting plural link elements is arranged, an arrangement is achieved that is advantageous in particular when forming a main body including a channel shaped interior cavity. Further advantageously the trigger surface can include a protrusion, in particular a hump that is detectable by a finger of a person using the arrangement.

In an advantageous embodiment of the invention the main body is approximately cuboid in a portion of the trigger mechanism, wherein two L-arrangements respectively including a trigger surface and plural link elements form a rectangle in combination in a cross section running orthogonal to the longitudinal axis of the main body. The cuboid shape elegantly facilitates the diametrically opposed arrangement of the trigger surfaces which facilitates ergonomic operability.

In order to simplify assembly, it is proposed that the main body includes two main body parts which are arranged at least substantially mirror symmetrical to a plane running through a longitudinal axis of the main element when the main element parts are assembled and/or which are arranged point symmetrical to this longitudinal axis in a sectional view, wherein the two main body parts are advantageously connected by positive form locking through snap hooks or interlocking lugs.

A particularly simple type of engagement between the sliding body and the at least one locking element is provided when the locking element contacts the proximal face of the sliding body in the operating position. Further advantageously the proximal face of the sliding body is formed by a socket section with a distal end of a tube inserted therein or by a radial shoulder at the sliding body.

The at least one locking element can contact a shoulder of the sliding body in the safety position in order to limit the pull back movement after triggering the actuation element. As an alternative to this double function of a locking element another contact element for the sliding body, e.g., also for a flange formed thereon can be provided at the main body. Advantageously the shoulder at the sliding body that provides a stop for the locking element in the safety position is formed by a flange that radially protrudes beyond an advantageously rotation symmetrical outer jacket of the sliding body.

According to another advantageous embodiment of the invention the flange includes a face at a distal end that forms a support surface for the actuation element that is advantageously configured as a coil spring. In this embodiment the flange performs a double function which simplifies the configuration of the sliding body. Particularly advantageously the actuation element is supported at a support surface of the first main body part with a distal end of the actuation element.

The cannula arrangement according to the invention has substantial advantages when mounting, in particular inserting the sliding body into the main body. For this purpose, initially the first main body part and adjacent thereto the second main body part are provided which are both arranged axially behind one another along the longitudinal axis of the main body or the sliding body, or the first and the second main body part are aligned in a linear manner when there is a film hinge connection between the first main body part and the second main body part. Then the sliding body and the actuation element are joined and inserted as a unit into the first main body part and the second main body part, or the actuation element without preload is inserted into the first main body part and the second main body part and the sliding body is only added in a next step which moves the actuation element into the preloaded condition.

In particular with respect to a safe and reliable positioning of the actuation element in the main body, the sleeve shaped first main body part has a big advantage. In particular when a coil spring is used as the actuation element, a coaxial alignment with the sleeve shaped first main body part can be performed and the actuation element is inserted so that it is secured at its distal end against sliding or escaping and jumping out reliably during the process of transitioning into the preloaded condition.

As a last step in the assembly sequence the third main body part is connected with the second main body part which completes the main body so it is provided in its final shape.

In order to simplify handling or in order to provide an alternative handling method in addition to gripping the housing shaped part of the main body it is proposed that the main body is provided with a respective gripping lobe at two opposite sides. The gripping lobes can be made from a rubber elastic material and squeezed against one another in a known manner by gripping with two fingers in a portion above the main body until they are in contact with one another.

According to an advantageous embodiment of the invention, the actuation element is supported with a distal end at a shoulder formed in an inner cavity of the first main body part or at a face wall terminating the first main body part in a distal direction. This assures a particularly simple and reliable fixing of the actuation element while moving the actuation element into the preloaded condition.

In another advantageous embodiment the gripping lobes can be connected to a sleeve shaped connector that is slid onto the main body. Thus, an overlapping portion between the connector and the main body envelops at least an axial section of the first main body part as well as a respective axial section of the second main body part and of the third main body part. The sleeve shaped connector thus not only causes a safe and firm connection of the gripping lobes at the main body but stabilizes the main body forming a jacket or a clamp ring which provides an additional safety in the assembled condition for the assembly of the main body from several main body parts.

A first embodiment of the first and second main body part, is characterized in that both parts are rigidly connected with one another. Thus, there is no pivotability between the two main body parts, in particular there is no film hinge between the main body parts. In this embodiment the alignment of the first body part with the second main body part is always automatically provided and a degree of freedom is eliminated during the assembly, wherein this degree of freedom would otherwise need to be eliminated by respective fixing and guiding the components.

As an alternative the embodiment recited supra, it is also possible to connect the first main body part and the second main body part with one another by a film hinge. In this case a hinge axle of the film hinge should be aligned perpendicular to and at a distance from the longitudinal axis of the sliding body.

With respect to the third main body part two basic cases of connection with the remaining main body parts are feasible. In a first case the third main body part and the second main body part can be connected with one another by a film hinge. The hinge axis of the film hinge can thus be aligned parallel or perpendicular to the longitudinal axis of the sliding body. In this variant the second main body part forms a center element of a three-element chain formed by the three main body parts which are connected by two film hinges, wherein the second main body part is connected with both film hinges.

As an alternative to the embodiment recited supra there is an option to connect the third main body part and the second main body part with one another by a film hinge. In this case like in the case where the first and the second main body part are connected, the hinge axis of the film hinge is aligned perpendicular to and at a distance from the longitudinal axis of the sliding body. In the latter case a symmetrical arrangement can be provided where the first main body part forms the center link of the three-element chain and the two end links of the chain are formed by advantageously identically configured main body parts. In this constellation, the second and the third main body parts are advantageously synchronously pivoted about the respective film hinge axes of the sliding body previously inserted into the first main body part and brought in contact with one another and fixated relative to one another.

For reasons of symmetry and in order to simplify assembly the two film hinges should have the same distance from the tip of the cannula viewed in the axial direction in the latter case. By the same token the two film hinges should have the same distance respectively from the longitudinal axis of the sliding body.

In an advantageous embodiment of the invention, the first main body part
is rotation symmetrical in a distal section, and/or
cuboid in a proximal section,
wherein a respective film hinge is advantageously arranged at opposite proximal edges of the cuboid proximal section. This embodiment facilitates a reliable arrangement of the distal end of the actuation element or sliding body in the rotation symmetrical distal section and a rotation safety of the sliding body in the cuboid proximal section if the sliding body has a cross sectional shape which prevents a rotation within the proximal cuboid section that advantageously has a rectangular cross section, in particular a square cross section.

According to an advantageous embodiment of the safety cannula arrangement according to the invention, the rotation symmetrical section of the first main body part has a cylindrical tip section and also has a cylindrical transition section configured to receive the actuation element that is compressed in the operating position, wherein the removable tubular cannula protection is advantageously slid onto the tip section.

According to an advantageous embodiment, the second main body part and the third main body part, in joined condition
form a gripping portion adjoining the film hinges in the proximal direction wherein the gripping portion is advantageously configured with ribs or a plurality of protrusions on an outer enveloping surface in order to improve gripping, and/or
form a trigger portion (36), adjoining the gripping portion in the proximal direction, wherein the trigger portion includes a trigger mechanism and/or
form a closure portion arranged at a proximal end of the main body wherein the second main body part and the third main body part are connected with one another by positive form locking through snap hooks and/or interlocking lugs.

According to an advantageous embodiment of the invention, the first main body part forms a distal end of the main body.

In an advantageous embodiment of the invention, the stop surface of the sliding body is arranged at the at least one rotation blocking body of the sliding body in order to limit the axial movement during transition of the sliding body into the safety position.

The sliding body according to the invention is thus characterized by a particularly simple configuration and economical manufacturability. In particular the invention is advantageously usable where the locking elements are not simultaneously used as rotation blocking bodies in order to fix the sliding body in the operating position. In many known safety cannula arrangements, the double function of the locking elements is provided. According to the invention the rotation blocking body can be formed by a separate component in addition to the locking elements of the trigger mechanism and can be typically arranged offset or advantageously axially offset from the locking elements. The rotation blocking body and the stop surface of the sliding body, however, are formed by the same component or are configured at the same component so that the double function provides an advantageous configuration with respect to installation space and complexity. The invention is variable with respect to the manner how the rotation blocking is achieved by the rotation blocking body of the sliding body. All geometries that prevent a rotation of the sliding body about its longitudinal axis while facilitating an axial movability of the sliding body in the main body for transfer into the safety position are conceivable.

According to an advantageous embodiment of the invention, the main body is provided with a respective gripping lug at two opposite sides, which improves handling or provides an alternative handling method in addition to handling the main body itself.

Ergonomics are further improved when a gripping portion for handling the device during puncture is advantageously exclusively arranged at the main body wherein ribs or a plurality of protrusions improving gripping are advantageously arranged in the grip portion. In particular this prevents that handling of the safety cannula arrangement also has to be performed in the portion of the cannula carrier which is disadvantageous because the cannula carrier moves relative to the main body when the trigger mechanism is activated.

Another embodiment of the invention proposes that the sliding body is configured as a hollow cylinder and the rotation blocking body of the sliding body is a protrusion or flange that protrudes radially outward beyond an outer enveloping surface of the hollow cylinder wherein the flange advantageously has a square circumference viewed in the direction of the longitudinal axis of the sliding body wherein corners of the circumference are advantageously rounded. The square shape of the circumference of the flange has advantages with respect to mounting the safety cannula arrangement since it facilitates an exact positioning of the cannula tip relative to the main body in a simple manner when the orientation of the cannula tip relative to the main body is known. Advantageously a protrusion or flange with square cross section cooperates with a square cross section in a section of the interior of the main body in order to achieve the required blocking against rotation. As a matter of principle, the protrusion or flange can also have a polygonal or elliptic shape.

In addition to the double function of a rotation blocking and configuration of a stop surface, the rotation blocking body arranged at the sliding body can also serve a third function. Namely, the actuation element can be supported at a distal face of the rotation blocking body arranged at the sliding body wherein the distal face is oriented opposite to the stop surface limiting the pull back movement. This makes the configuration particularly simple and economically producible.

According to an embodiment of the safety cannula arrangement according to the invention the stop surface of the base element can be formed by a radially inward extending protrusion that is formed at a wall of the main body that envelops the interior of the main body. The protrusion can be formed by a shoulder in the main body wall or a typically distal main body face wall which can be also merely a partial face wall.

Last not least, a particularly advantageous embodiment includes two protrusions that are arranged diametrically opposite to one another and which respectively have a triangular shape viewed in the direction of the longitudinal axis of the sliding body, in particular with an arcuate cambered contour wherein each protrusion advantageously connects two wall sections of the main body in a corner portion wherein the wall sections are arranged relative to each other in a L-shaped cross section. In a main body that is divided in two parts in the portion of the rotation blocking device, the protrusions serving as stops also perform a stiffening function preventing an unintentional deformation of the L-shaped wall sections that are connected with one another and thus facilitate maintaining the rectangular cross section of the main body in this area.

According to an advantageous embodiment a maximum diameter or a maximum width of the sliding body extending perpendicular to the longitudinal axis of the sliding body in a exit section of the sliding body that adjoins the proximal end of the main body in the proximal direction is 80% at the most, advantageously 70% at the most, further advantageously 60% at the most of a maximum diameter of the main body or a maximum width of the main body extending perpendicular to the longitudinal axis of the sliding body when the sliding body is provided in the safety position.

The invention thus provides a particularly slender sliding body that has little resistance against the pull back movement so that a risk of the sliding body getting hooked at the skin of the patient or at other objects which would slow down the pull back movement out of the main body is particularly low. In particular no elastically deformable locking elements like spring elastic tongues or similar are provided at the sliding body according to the invention, which would create a particular risk of entanglement. Therefore, the safety cannula arrangement according to the instant invention assures a correct pull back movement of the sliding body after completion of the blood draw or infusion with a very high probability so that pain or trauma in the patient are prevented in the best way possible.

The arrangement of the locking elements in the interior of the main body furthermore prevents an unintentional triggering of the pull back mechanism of the sliding body. Furthermore, arranging the trigger members and the locking elements of the trigger mechanism at the main body facilitates a simple configuration of the sliding body and ergonomic operability when triggering the pull back movement.

In order to provide a maximum stability of the free cross section of the cannula in the operating position of the safety cannula arrangement a distance measured along the longitudinal axis of between a bearing arranged at a maximum distance in the distal direction and a bearing of the sliding body arranged at a maximum distance in the proximal direction is at least 100%, advantageously at least 120%, further advantageously at least 130% and even more advantageously at least 160% of a length of the main body measured in a direction of the longitudinal axis wherein the bearings are respectively suited to react transversal forces impacting the sliding body, wherein the transversal forces run in any radial direction with respect to the longitudinal axis of the cannula or of the main body. A large bearing spacing provides good reaction of transversal forces from the sliding body into the main body so that the reception and support of the sliding body in the main body can be provided with particularly little clearance. This in turn provides a particularly advantageous function when puncturing skin since transversal forces imparted by the user through the main body and the sliding body upon the cannula are transferrable very directly and without elasticity or rocking into the puncturing location and in particular upon the blood vessel to be punctured. This provides a system with a high level of stiffness in the transversal direction which improves on target puncturing. This applies in particular for so called rolling veins which are characterized in that they try to escape laterally from a transversal force imparted by the cannula before puncturing. The stiffer the entire system in a direction transversal to the longitudinal axis of the sliding body and the smaller the clearance in this direction, the higher the possibility that the puncture can actually be performed at the intended spot.

Furthermore it is advantageous that a distance between a bearing that is arranged at a maximum distance in the distal direction and a bearing of the sliding body arranged at a maximum distance in the proximal direction is at least 70%, advantageously at least 80%, further advantageously at least 90%, even more advantageously at least 95% of a length of the sliding body measured in the direction of the longitudinal axis, wherein the bearings are respectively suited to react transversal forces impacting the sliding body and running in any radial direction. The considerations and advantages with respect to bearing spacing with respect to main body length recited supra apply. Typically, the main body length is greater than the sliding body length since at least one joint between a proximal end of the cannula holder and a distal end of the tube is provided within the main body.

In another advantageous embodiment of the invention the bearing point that is arranged at the largest distance in the distal direction and/or the baring point that is arranged at the largest distance in the proximal direction join directly at the distal or proximal end of the sliding body and/or extend to this end. In this embodiment the entire length of the sliding body is used as bearing point distance, this means as a support length so that a particularly high level of stiffness or stability of the system is provided under transversal force impact.

According to an advantageous embodiment of the invention the baring point arranged at a proximal end of the sliding body, advantageously the bearing point arranged at the largest distance in the proximal direction is formed by at least one protrusion of the main body extending into an inner cavity of the main body and forming a contact surface limiting the movement of the sliding body from the operating position into the safety position. In this case a particularly advantageous double function of the protrusion at the main body is provided which simplifies the configuration.

When the sliding body does not include elastically deformable elements or members which cooperate when the trigger mechanism is activated and/or which are displaceable when the trigger mechanism is activated this provides not only a particularly simple embodiment of the sliding body but also runs the risk that a sliding body section exiting from the main body when the sliding body is displaced into the safety position gets entangled at objects or the skin of the patient which impedes the pull back movement.

Last but not least, an advantageous embodiment of the invention arranges the trigger mechanism at two base body parts so that those two base body parts jointly form the trigger mechanism wherein the two base body parts are joined by a movement relative to each other wherein the movement has a movement component perpendicular to the longitudinal axis of the sliding body. Contrary to the prior art represented by WO 2016/007438 A1 the joining of two main body parts in this embodiment is not performed by axial insertion or plugging them into each other, but by a movement transversal or perpendicular to the sliding body longitudinal axis or by a pivoting movement about a pivot axis perpendicular to the longitudinal axis which can be the case in particular when forming at least one film hinge between two main body parts.

### EMBODIMENTS

The invention is subsequently described based on an embodiment with reference to drawing figures, wherein

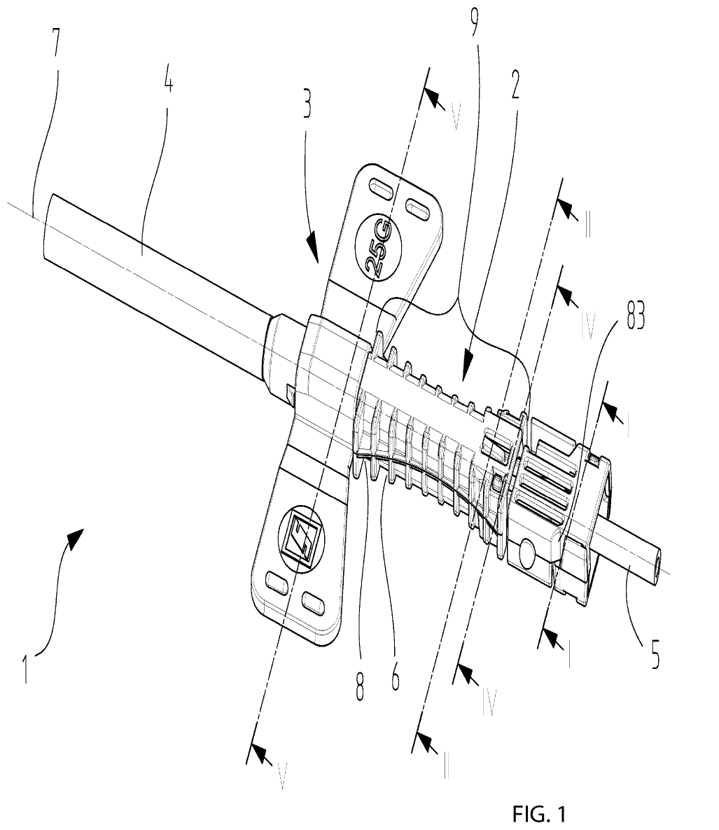

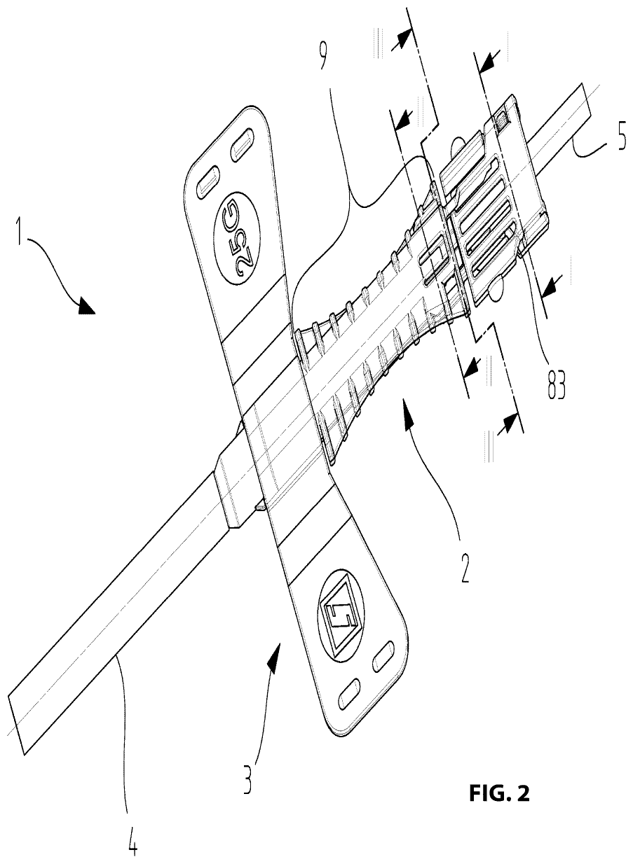

FIGS. 1 and 2: illustrate a safety cannula arrangement in two perspective views including a cannula protection applied at a distal end;

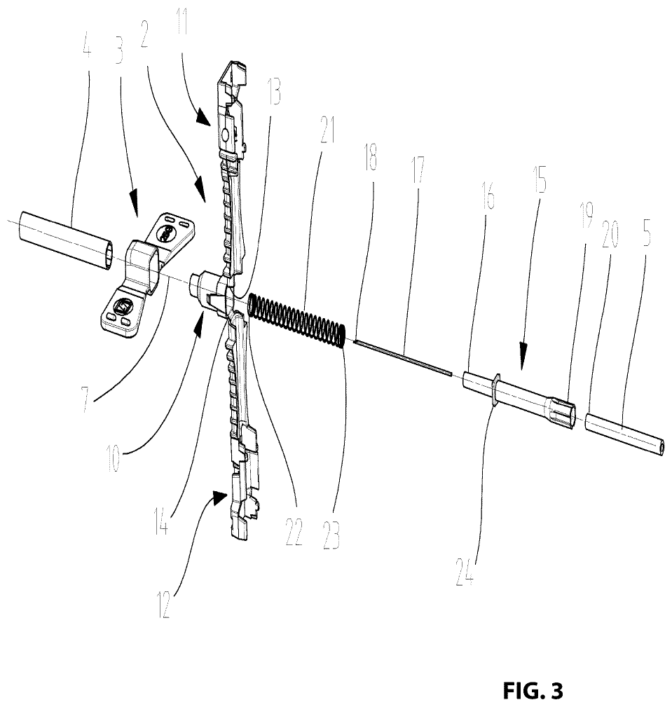

FIG. 3: illustrates an exploded view of the safety cannula arrangement;

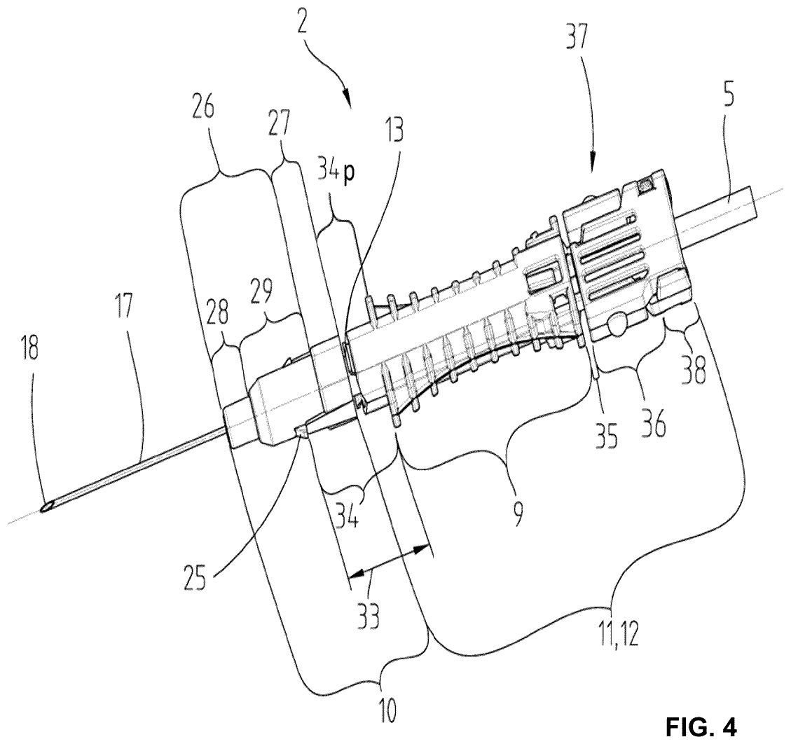

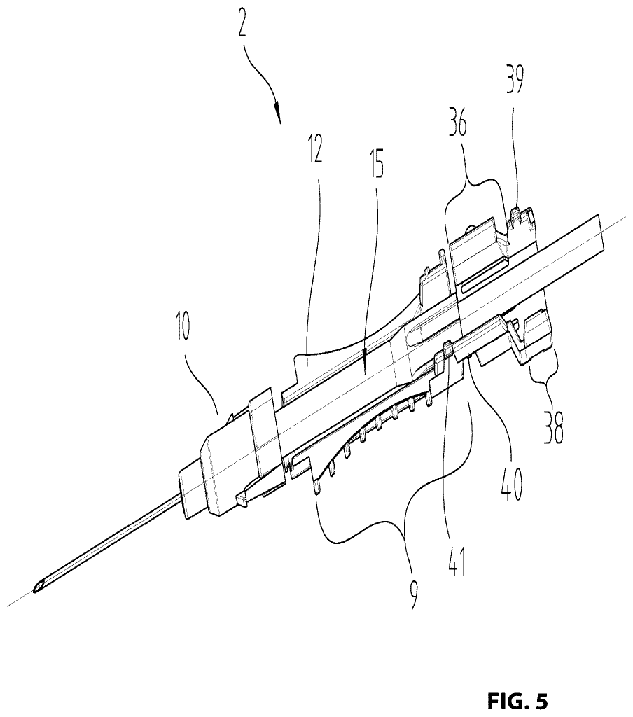

FIG. 4: illustrates a perspective view of the main body including the cannula protruding there from;

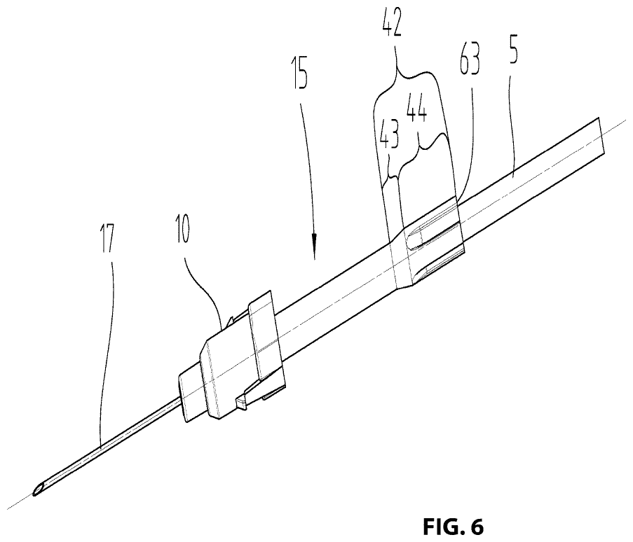

FIG. 5: is similar to FIG. 4 but shows a section of the main body;

FIG. 6: illustrates a released first main body part including a sliding body inserted therein;

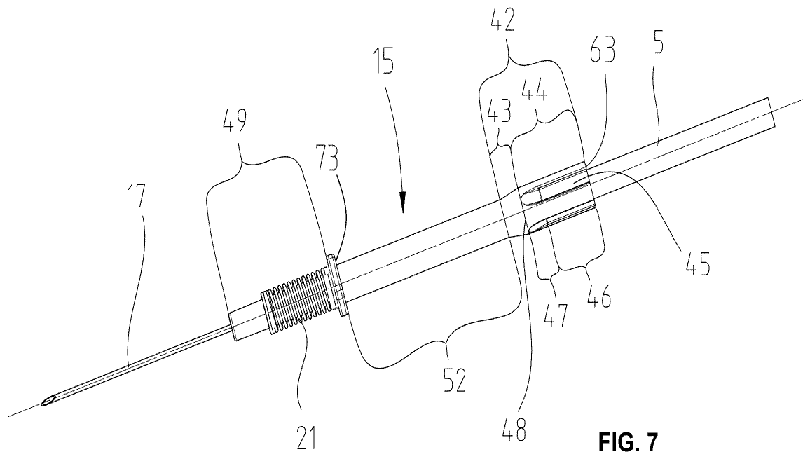

FIG. 7: illustrates the sliding body with a cannula, actuation element and a section of a flexible tube;

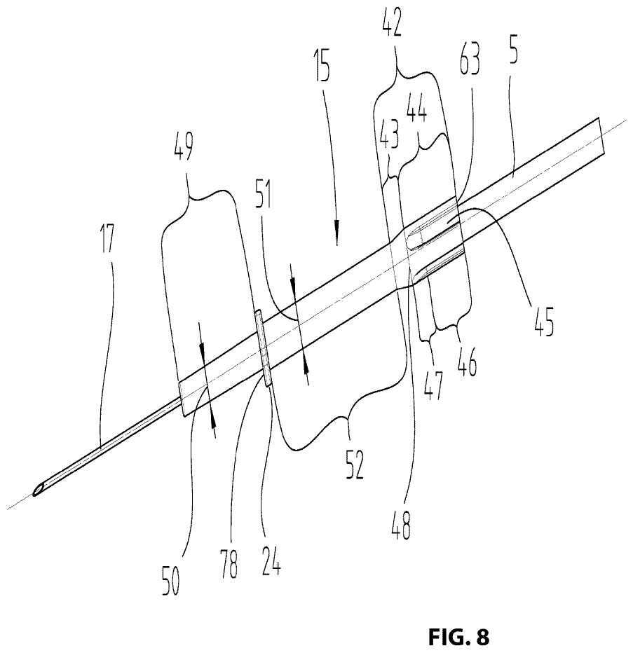

FIG. 8: is similar to FIG. 7, but does not include the actuation element;

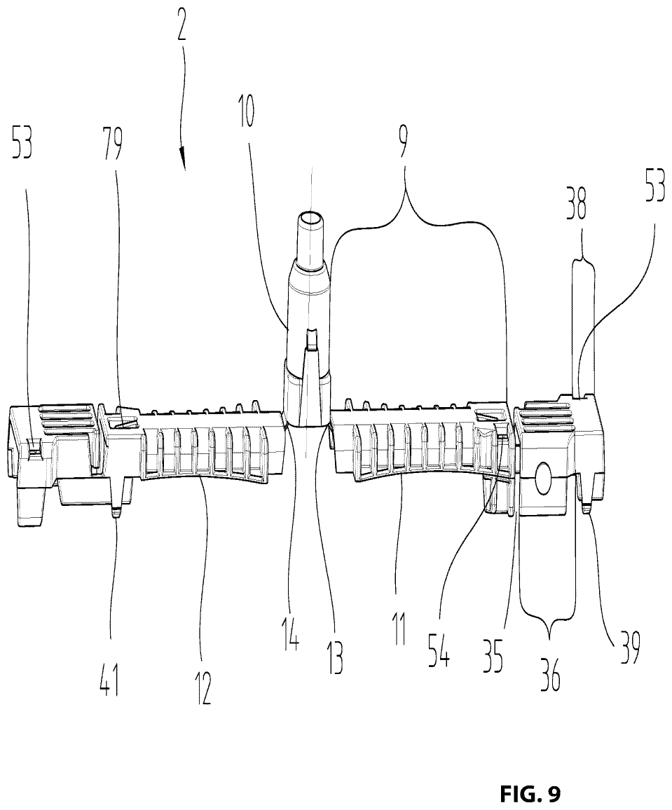

FIG. 9: illustrates the perspective view of the main body including a second and third main body part folded out laterally;

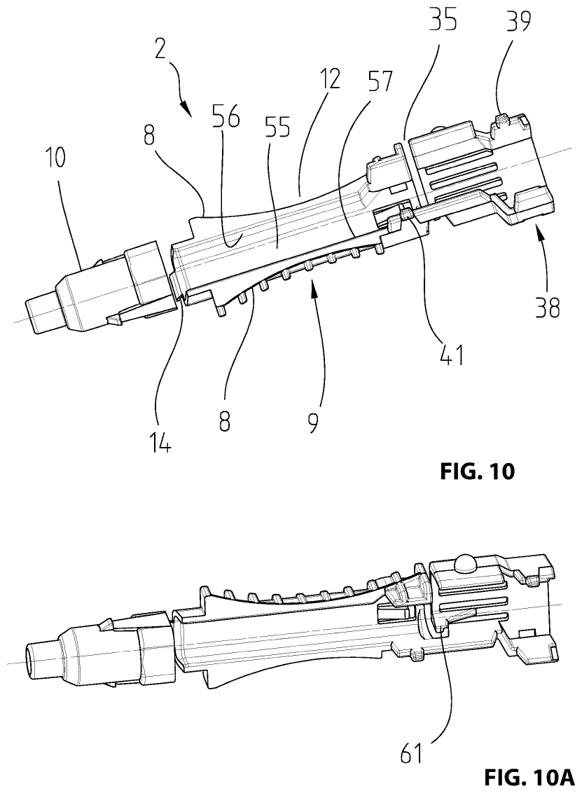

FIG. 10: illustrates a perspective view of the first and second main body part;

FIG. 10A: is similar to FIG. 10, but shows a different perspective;

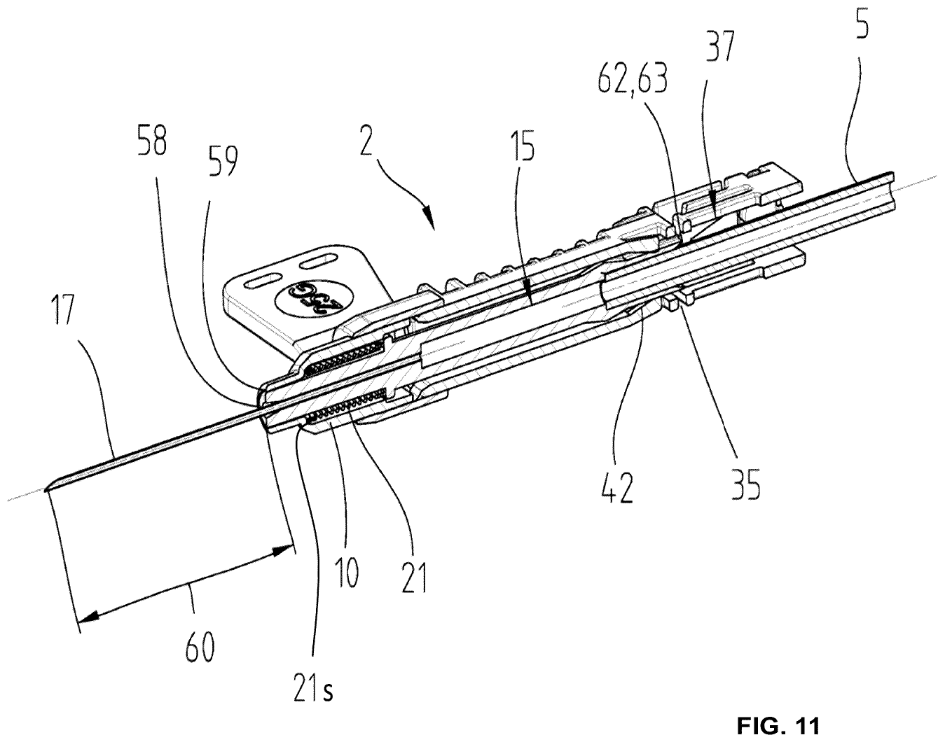

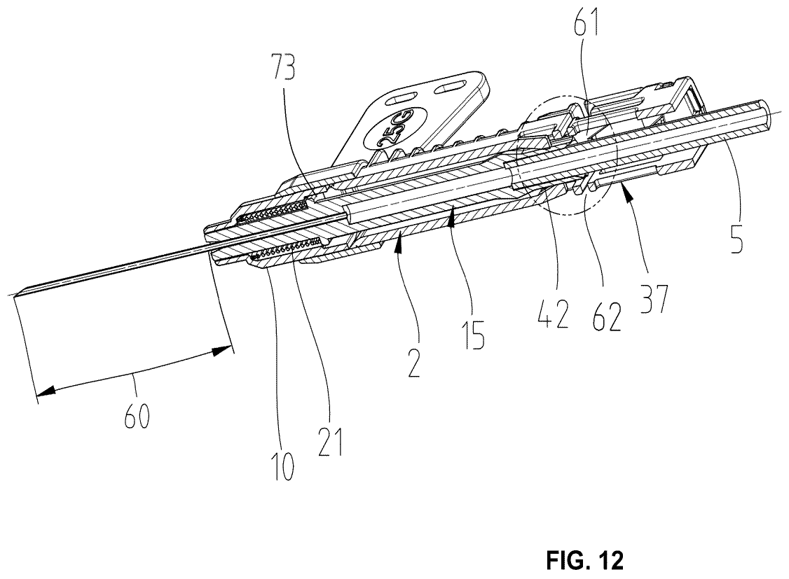

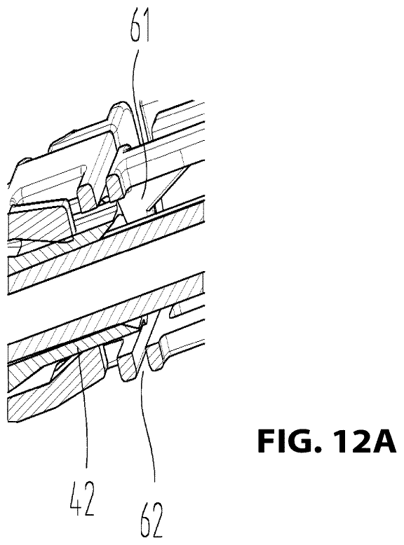

FIGS. 11 and 12: illustrate a longitudinal sectional view according to FIGS. 1 and 2 showing the sliding body in the operating position;

FIG. 12A: illustrates a blown-up detail of FIG. 12

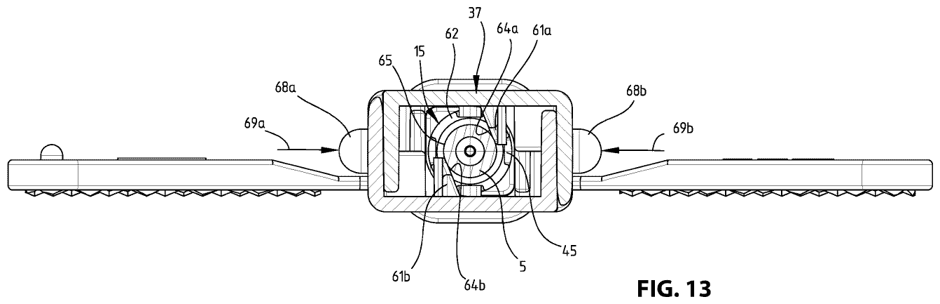

FIGS. 13 through 16: illustrate a sectional view along the line I-I through the safety cannula arrangement according to FIGS. 1 and 2 in different positions of the trigger members and locking elements;

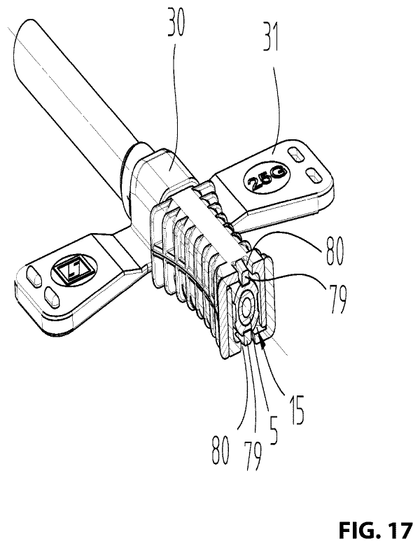

FIG. 17: illustrates a perspective view of the safety needle in a sectional view along the line II-II in FIGS. 1 and 2;

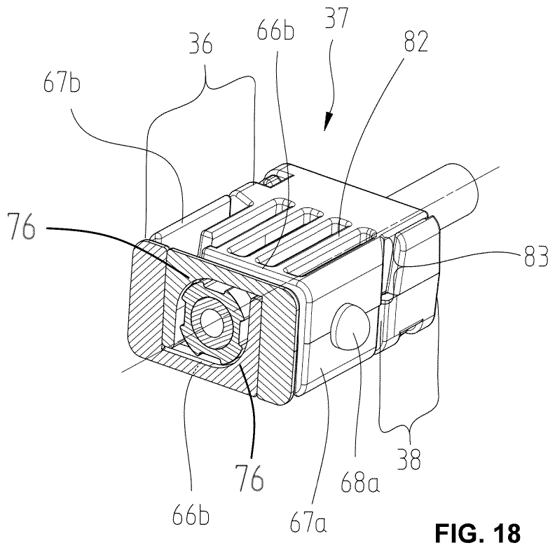

FIG. 18: illustrates a trigger mechanism in a sectional view along the line III-III like in FIG. 1 looking in the proximal direction;

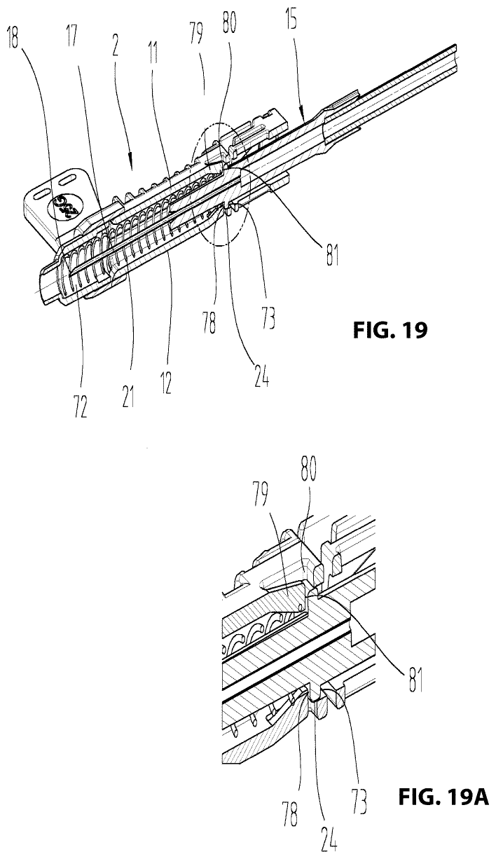

FIG. 19: illustrates a longitudinal sectional view through the safety needle arrangement according to FIGS. 1 and 2 in the safety position of the sliding body;

FIG. 19A: illustrates a blown-up detail of FIG. 19;

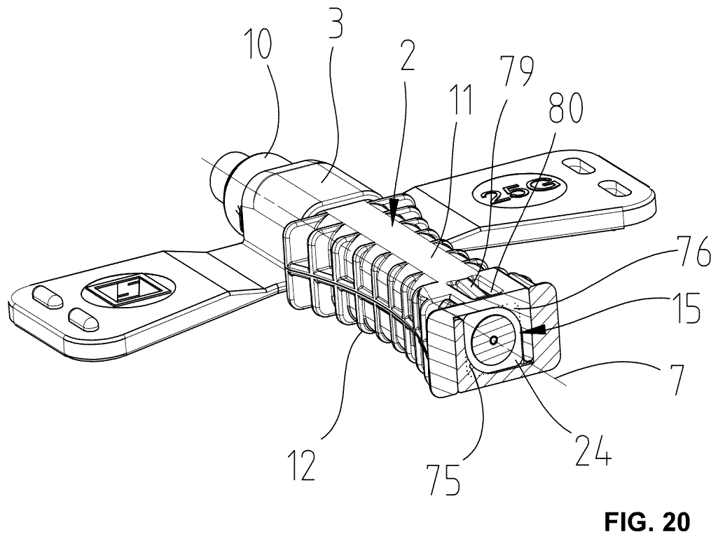

FIG. 20: Illustrates a sectional view of the safety cannula arrangement according to FIGS. 1 and 2 along the sectional like IV-IV with the sliding body in the safety position in the distal viewing direction;

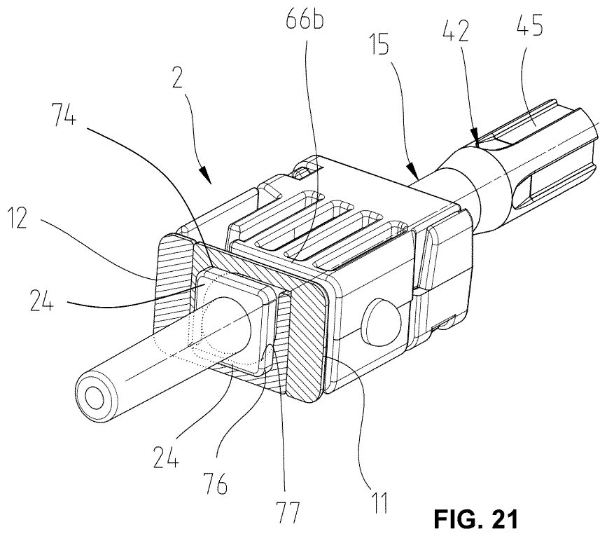

FIG. 21: is similar to FIG. 18, however shows the safety position of the sliding body; and

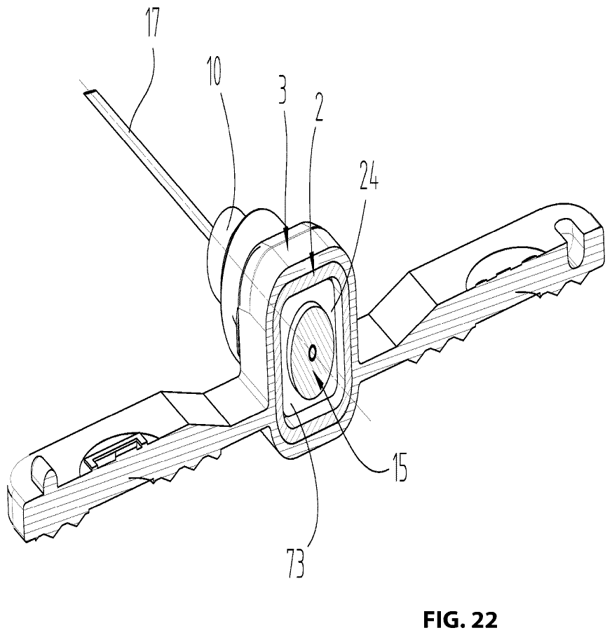

FIG. 22: Illustrates a cross sectional view of the safety cannula arrangement according to FIGS. 1 and 2 along the line V-V in the distal direction.

FIGS. 1 and 2 show a safety cannula assembly 1 in different perspectives in shipped configuration including a main body 2 to having an elongated housing, a lobe module 3 arranged at the main body, a tubular cannula protection 4 slid over a distally protruding cannula and a flexible tube 5 that is cut off for illustration purposes. The cut-off tube 5 that is connected at a proximal end section of a sliding body that is not illustrated in FIGS. 1 and 2 has a length of 5 cm to 30 cm and includes an adapter at a proximal end wherein the adapter establishes connectivity with additional handling devices used for blood drawing. The main body 2 includes a plurality of transversal ribs 6 running perpendicular to a longitudinal axis 7 of the main body or also of the entire safety cannula assembly including the sliding body, wherein a protrusion of the transversal ribs beyond the rounded cuboid base geometry of the grip portion 9 of the main body 2 initially decreases from a maximum protrusion at the transversal ribs 6 adjacent to the lobe module 3 towards the proximal end of the grip portion of the main body 2 and subsequently increases again. The transversal ribs 6 running equidistant and parallel to one another are connected with one another and stabilized at each side by a longitudinal rib 8 arranged in a symmetry plane of the main body 2. This way a particularly safe and ergonomic grip is facilitated for two fingers of a person using the safety cannula arrangement 1 wherein the two fingers grip the grip portion 9 of the main body from opposite sides which facilitates handling.

It is evident from the exploded view according to FIG. 3 that the main body 2 is assembled from three main body parts 10, 11 and 12. Thus, from a sleeve shaped first main body part 10 which extends the most in the distal direction and two half shell shaped main body parts including the second main body part 11 and the third main body part 12. The second main body part 11 and third 12 are respectively connected with the first main body part 10 by a film hinge 13, 14, and thus pivotally movable about a respective hinge axis of the film hinge 13, 14 relative to the first main body part 10. The two main body parts 11 and 12 are identically configured but arranged symmetrical relative to a symmetrical plane which is parallel to the two film hinges 13, 14 and runs through the longitudinal axis 7 of the main body.

The inner cavity of the main body 2 includes an essentially hollow cylindrical sliding body 15 that is movably supported in the axial direction, this means in the direction of the longitudinal axis 7 relative to the main body 2. A cannula 17 including a tip 18 formed at a distal end by a ground surface is inserted into the sliding body and sealed therein. A proximal end section 19 that is formed as a socket section is inserted with a distal end section of the tube 5 which is sealed therein. Therefore, the sliding body 15, The cannula 17, and the tube 5 form a firmly connected unit whose components besides the flexibility of the tube 5 are not movable relative to one another. An activation element 21 configured as a coil spring is externally slid onto the distal end section 16 of the sliding body 15 in order to support the sliding body 15 in the operating position under a preload in the main body 2 and in order to provide a propulsion for a pullback movement of the sliding body 15 relative to the main body 2 in the proximal direction, wherein the coil spring is supported with a distal end 22 in the first main body party 10 and supported with a proximal end 23 at a flange 24 that has a rectangular cross section and that protrudes beyond an outer enveloping surface of the sliding body 15.

Subsequently the configuration of the main body 2 made from main body parts 10-12 and the cooperation of the main body 2 with the sliding body 15 is described in more detail with reference to FIGS. 4 through 10.

It is evident from FIG. 4 that the main body 2 can be divided into different portions in the axial direction. Starting at a distal end of the main body 2 the sleeve shaped first main body part 10 includes a rotation section that is rotation symmetrical besides two interlocking lugs 25 for fixing the lobe module 26 and an approximately cuboid section 27 adjacent in the proximal direction. The rotation symmetrical section 26 is divided into a cylindrical tip section 28 onto which the cannula protector 4 not illustrated in FIG. 4 but illustrated in FIGS. 1 and 2 can be slid and a cylindrical transition section 29 that has a conical taper at a distal end and that includes a distal end section 16 of the sliding body 15 including an actuation element 21 slid thereon. As evident from FIGS. 3, 1 and 2 the lobe module 3 is slid onto the main body 2 from a distal end of the main body with a center connection part 30 of the lobe module 3 where a respective gripping lobe 31 is integrally provided at opposite longitudinal sides of the lobe module 3 as evident from FIG. 17. An axially measured length 32 of the connection part 31 of the lobe module 3 thus corresponds to a length 33 of an overlapping portion 34 that extends over the first main body part 10 and also over the joined second main body part 11 and the third main body part 12 and that can be divided into the sections 34d and 34p wherein the first distal section 34d overlaps the first main body part 10 and the proximal section 34p overlaps the second and the third main body part 11, 12. The overlapping portion 34 includes an entirety of the cuboid section 27 in the portion of the first main body part 10 and includes a portion of the substantially cylindrical transition section 29, thus up to the interlocking lugs 25 included therein. The connection part 30 of the lobe module 3 thus bridges in particular also the portion of the two film hinges 13, 14 and a gap portion included therein between the first main body part 10 and the second main body part 11 and the third main body part 12. Starting from a proximal end of the overlapping portion 34 the grip portion 9 described supra adjoins in the proximal direction wherein the grip portion is jointly formed by the two main body parts 11 and 12. A circumferential notched portion 35 is arranged further in the proximal direction and a trigger portion 36 adjoins further in the proximal direction and includes a trigger mechanism 37 that will be described in more detail infra. A proximal end section of the main body 2 is formed by a closure portion 38 where the second main body part 11 and the third main body part 12 are connected with one another by snap hooks by positive form locking. An opening of the main body 2 provided by a pivot movement of the two main body parts 11 and 12 is thus prevented on the one hand side by the snap hooks in the closure position 38 and on the other hand side by the connector 30 of the lobe module 3 that is slid over.

FIG. 5 shows how the sliding body 15 is arranged in the interior cavity of the main body 2 not showing the second main body part 11 but only the lower third main body part 12. The distal end section 16 of the sliding body 15 and the actuation element 21 are not shown because they are arranged covered in the sleeve shaped first main body part 10. The closure portion 38 shows a snap hook 39 that cooperates with an accordingly adapted recess in the second main body part 11 in a joined condition of the two main body parts 11 and 12 and that forms a closure that cannot be disengaged without destruction. The trigger portion 36 and the grip portion 9 are exclusively connected by a connection bar 40 in each of the two main body parts 11, 12 wherein the connection bar is connected with its proximal end with the grip portion 9 and connected with the trigger portion 36 at its distal end. It is appreciated that the entire second and third main body part 11, 12 and the main body 2 made from the three main body parts 10, 11, 12 including the film hinges 13, 14 is produced as one integral injection molded component. FIG. 5 shows an additional snap hook 41 at the main body part 12, wherein the snap hook 41 is arranged in the grip portion 9 and also cooperates with an accordingly adapted recess in the second main body part 11 and keeps the two main body parts 11 and 12 together in addition to the snap hooks 39 and the connector 30 of the lobe module 3.

FIG. 6 shows the sliding body 15 including the cannula 17 inserted therein and the tube 5 inserted in the proximal end additionally showing only the first main body part 10. The sliding body 15 includes a socket section 42 that has a greater diameter than the center section and which adjoins in the proximal direction wherein the socket section 42 is formed by a shorter transition section 43 and a substantially cylindrical insertion section 44 adjoining the proximal direction. The insertion section 44 includes an inner cylindrical bore hole that is adapted to an outer diameter of the flexible tube 5 and into which the tube is glued, tightly sealed.

It is evident from FIG. 7 that the socket section 42 includes four stress relief grooves evenly spaced over the circumference of the socket section 42 and running in a direction of the longitudinal axis of the sliding body 15 for blocking tongues of the main body that will be described infra. The stress relief grooves 45 include a longer section 46 where they have a greater depth and a groove base that runs parallel to the longitudinal axis 7 and a shorter distal section 47 where the groove base rises in a ramp to a circumferential border line between the insertion portion 44 and the conical transition section 43. The function of the stress relief grooves in combination with the blocking tongues recited supra will the described infra.

FIG. 7 illustrates the compressed actuation element 21 on a distal end section 49 of the sliding body 15 which is not the case in the otherwise analogous representation of FIG. 8. This indicates that a diameter 50 in the distal end section 49 of the sliding body 15 is greater than a diameter 51 in a center section 52 of the sliding body 15.

The individual portions of the main body 2 and the mirror symmetrical arrangement of the identically formed main body parts 11 and 12 are evident from FIG. 9 where the main body 2 is shown with the main body parts 11 and 12 laterally folded out from the first main body part 10. In this condition the main body 2 is removed as an injection molded component from the injection molding tool. The grip portion 9, the adjacent notch portion 35, the adjoining trigger portion 36 and the closure portion 38 forming the proximal closure are evident from FIG. 9. A recess 53 is visible on the third main body part 12, shown on the left side of FIG. 9, wherein the snap hook 39 is receivable and lockable in the recess 53 as evident from FIG. 5. Another recess 54 in the grip portion 9 is shown and provides locking reception of the corresponding snap hook 41 at the grip portion 9 of the third main body part 12. Thus, there is a total of two snap hooks 39 and two recesses 53 in the closure portion 38 and two snap hooks 41 and two recesses 54 in the grip portion 9. Each of the two main body parts 11 and 12 includes a respective snap hook 39, 41 and a respective recess 53, 54 in the grip portion 9 as well as in the closure portion 38.

FIG. 10 shows a view into the groove shaped third main body part 12 since the second groove shaped main body 11 part was removed which would otherwise be arranged there above in an assembled condition and on the other hand side also the sliding body 15 and the cannula 17 and the tube 5 are not inserted. It is evident that a base 55 and two opposite half-walls 56, 57 of the main body part 12 are arranged perpendicular to one another which yields a square cross section of the interior cavity when the two main body parts 11, 12 are assembled. The two longitudinal ribs 8 of the main body part 12 arranged on opposite sides are jointly formed by both main body parts 11 and 12 in assembled condition and define a center plane in a contact portion of the two main body parts 11, 12 wherein the center plane runs parallel to the longitudinal axis 7 and also forms a plane of symmetry. FIG. 10 also shows the snap hooks 41 in the grip portion 9 and 39 in the closure portion 38 wherein the snap hooks are arranged on opposite sides in respect to the longitudinal axis 7

FIGS. 11, 12 and 12A respectively show a longitudinal sectional view of the safety cannula arrangement 1 in an operating condition of the sliding body 15 in different perspectives. A distal face 58 of the sliding body 15 thus eventually terminates flush with a distal face 59 of the first main body part. The cannula 17 protrudes with a free length 60 beyond the distal face 59 of the base body part 10. The actuation element 21 is in a preloaded condition so that the sliding body 15 tends to move relative to the main body 2 in the proximal direction. This movement is prevented by two locking elements 61a, 61b which form part of the trigger mechanism 37 and cooperate with a proximal face 62 of the sliding body 15 in its socket section 42. The face 62 of the sliding body 15 forms a shoulder 63 in combination with the tube 5 that has a smaller diameter which is evident from FIGS. 6 through 8. This shoulder is provided in the operating position 15 viewed in the axial direction in the notched portion 35 at its proximal end that is defined by the locking elements 61a, 61b of the trigger mechanism 37. The sliding body 15 is thus fixed without clearance in the axial direction in the main body 2 in the operating position which is indispensable for performing the puncture correctly.

Viewing FIGS. 13 through 16 and 18 together makes the function of the trigger mechanism 37 evident. The proximal face 62 that forms the shoulder 63 in the transition from the tube 5 to the socket section 42 of the sliding body 15 is retained by two diametrically opposed locking elements 61a, 61b, c.f. also FIG. 10A, so that the sliding body 15 remains in the operating position 45. The socket section 42 of the sliding body 15 includes relief grooves which yields a circular contour of the face 62 in a cross section wherein, the circular contour is interrupted by four indentations having a 90-degree offset.

The locking elements 61a, 61b have a triangular or trapezoid shape in an axial viewing direction and contact an outer enveloping surface 65 of the tube 5 with a respective control edge 64a, 64b.

It is evident from FIG. 18 that the locking elements 61a, 61b are coupled respectively by a compression rod 66a, 66b with a trigger member 67a, 67b. The trigger members 67a, 67b are respectably provided with a protrusion 68a, 68b configured as a semi spherical button. The trigger members 67a, 67b form two approximately square surfaces in a side view of the main body 2, the square surfaces having a square protrusion 68a, 68b wherein the compression rods 66a, 66b run at an angle of 90 degrees relative thereto. Thus, the locking elements 61a, 61b run at an angle of approximately 90 degrees relative to the compression rods 66a, 66b so that a C-shape or U-shape is achieved overall. Thus, the trigger members 67a, 67b and the locking elements 61a, 61b respectively form a free arm of the U or C, whereas the compression rods 66a, 66b form a base element of the U or C.

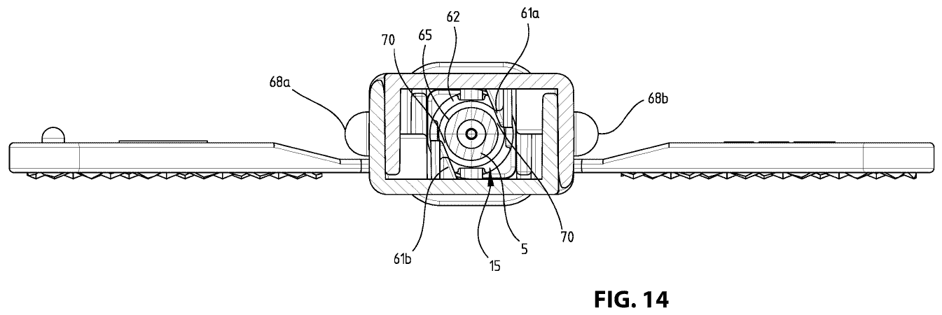

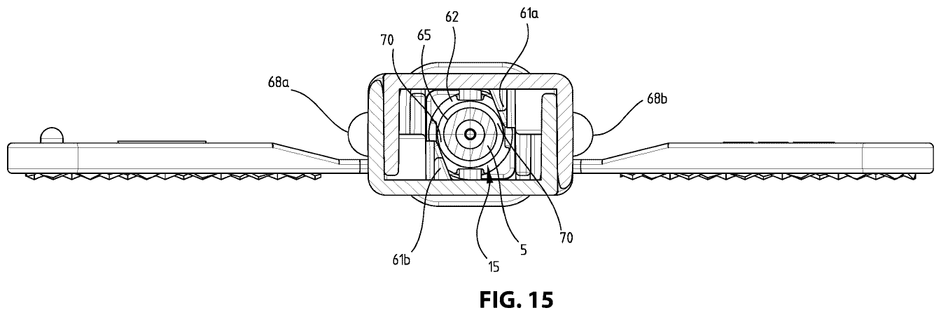

When the sliding body 15 needs to be transferred into the safety position after completing the blood draw or the infusion with the safety cannula arrangement 1 so that the cannula 17 is arranged within the main body 2 in its entirety, the operator of the safety cannula arrangement 1 imparts a pressure force upon the protrusions 68a, 68b of the trigger members 67a, 67a wherein the pressure force is imparted with two fingers of one hand simultaneously and runs in a radial direction according to the arrows 69a, 69b. Starting from the locking position illustrated in FIG. 13 where the locking elements 61a, 61b block the sliding body 15 by an engagement (contact at the face 62) so that the upper locking element 61a in FIG. 13 moves to the right due to pressure upon the left trigger member 68a whereas the lower locking element 61b in FIG. 13 moves to the left due to pressure upon the right trigger member 68b. FIG. 14 shows a condition where a distance 70 has formed between the locking elements 61a, 61b and the outer enveloping surface 65 of the tube 5 due to a displacement of the trigger members 68a, 68b forming a rigid unit and of the locking elements 61a, 61b connected therewith. This distance 70, however, is not large enough yet to release a contact of the face 62 at the locking elements 61a, 61b. Therefore, the cannular carrier 15 remains in the operating position in this position of the trigger mechanism or the trigger members 67a, 67b. FIG. 15 shows another intermediary state where the distances 70 between the locking elements 61a, 61b and the enveloping surface 65 of the tube 5 are larger but still cause an overlap between the locking elements 61a, 61b and the face 62 of the sliding body 15 and thus a blocking of the sliding body 15.

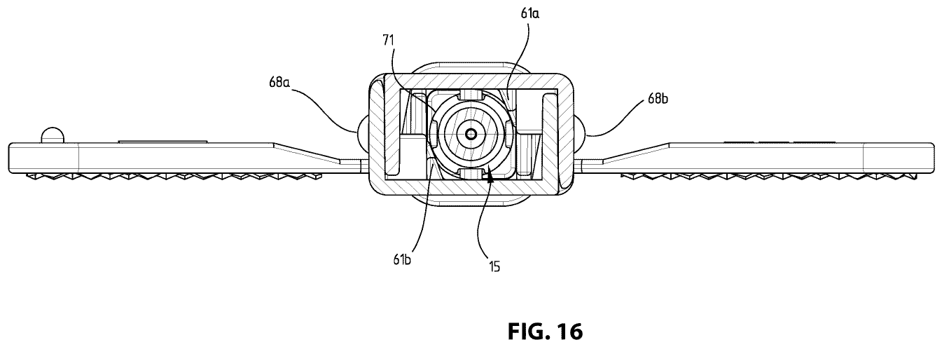

When the trigger members 67a, 67b with their associated knob shaped protrusions 68a, 68b are in the position shown in FIG. 16 the associated locking elements 61a, 62b are displaced outward enough so that the cross section of the sliding body 15 defined by a circular enveloping line 71 is released in its entirety in the portion of the socket section 42. Now the locking elements 61a, 61b are out of engagement with the sliding body 15 so that the sliding body 15 can move in the proximal direction driven by the actuation element 21 which causes a pull back of the cannula 17 into the interior of the main body 2.

Accordingly, FIGS. 19 and 19A show the sliding body 15 in the safety position where the cannula 17 including the tip 18 is arranged within an interior cavity 72 of the main body 2. The actuation element 21 configured as the coil spring is now provided in a condition where it has less tension in the operating position of the sliding body 15.

In order to prevent an exit of the entire sliding body 15 and thus also of the cannula 17 at the proximal end of the main body 2 the safety position of the sliding body 15 is defined by a stop surface 73 arranged at the sliding body 15. The stop surface 73 arranged at the sliding body 15 thus cooperates with a stop surface 74 of the main body 2 that is formed at the proximal end of the main body 2, thus at the proximal end of the closure portion 38 which is formed jointly by the second main body part 11 and the third main body part 12.

It is evident from the sectional view of FIG. 20 that the second main body part 11 and the third main body part 12 are U-shaped in cross section wherein the two U-shaped cross sections of the walls are nested into one another in a direction perpendicular to the plane of symmetry of the main body 2, this means in a direction perpendicular to the two film hinges, this means of the pivoting motion during the joining process and thus define an essentially square free cross section in the interior cavity 72 of the main body 2. The cross-section shape in the portion of the stop surfaces 74 of the main body parts 11 and 12, however is not exactly square but two corner portions are filled and defined towards the longitudinal axis 7 of the main body 2 respectively by an arcuate line 75 which forms a quarter circle in the respective corner of the U or C. FIG. 20 shows the edges of an imaginary square where the stop segments 76 defined by the arcuate line 75 are introduced into the corners in dashed lines for the purpose of clarity.

As evident from FIG. 20, the radius of a circle that runs through the slightly rounded corners of the square flange 24, is greater viewed in the direction of the longitudinal axis 7 than a radius of a circle which is formed by a continuation of the two arcuate lines 75. Therefore, the square flange 24, cannot pass through the free cross section in the area of the stop surfaces 74 that are formed at the stop segments 76 so that a displacement of the sliding body 15 stops at this location. This is also clearly evident from the sectional view according to FIG. 21. The square flange 24 contacts the stop surfaces 76 of the main body parts 11, 12 in the portion of the rounded sections 77. The cannula carrier and sliding body 15 is in the safety position in the condition illustrated in FIG. 21 where the socket section 42 has exited from the main body 2 at a proximal end of the sliding body 15.

It is evident from FIG. 22 that the interior cavity 72 in the cuboid section 27 of the overall sleeve shaped first main body part 10 is square with rounded corners. The cross section of the square flange 24 is adapted to this cross-section shape so that a smooth low resistance movement of the sliding body 15 is facilitated in the axial direction under an impact of the force of the actuation element 21 and a rotation of the sliding body 15 about the longitudinal axis 7 is reliably prevented. Thus, the flange 24 performs the function of a rotation blocking body of the sliding body 15 and the corresponding cuboid section 27 of the first main body part 10 forms a rotation blocking body of the main body 2. While the proximal contact surface 73 is arranged at the square flange 24 of the sliding body 15 defining the pull back movement of the sliding body 15 the square flange 24 has a double function since its additional stop surface 78 that is not shown in FIG. 22, but shown in FIGS. 7 and 8 supports the actuation element 21.

In addition to limiting the pull back movement of the sliding body 15 in the proximal direction it is also important to prevent that the sliding body 15, after having moved into the safety position, is moved back in the distal direction intentionally or unintentionally so that the cannula 17 is exposed again and the safety cannula arrangement 1 may be used again which shall be avoided and prevented under all circumstances. The blocking tongues 79 recited supra serve this purpose wherein the blocking tongues are configured as spring elastic elements of the main body 2 and respectively arranged within a window 80 enveloping the blocking tongues 79 on three sides. This configuration is clearly evident from FIGS. 19 and 19A, where the sliding body 15 is in the safety position. A respective face 81 of the blocking tongue 79 contacts the distally oriented contact surface 78 of the square flange 24 so that the square flange 24 performs three functions: end stop for the pull back movement, support of the actuation element 21 and stop surface for the blocking tongues 79. Since the blocking tongues 79 are elastically connected with the respective main body part 11, 12 the blocking tongues 79 move elastically radially outward when the sliding body 15 passes through during the pull back movement and move back radially inward after the passage of the square flange 24 due to the preload of the blocking tongues 79 and perform the blocking in cooperation with the flange 24. Since the diameter of the sliding body 15 in the distal end section 49 is smaller than in the center portion 52 a sufficiently large overlap of the blocking tongues 79 with the protrusion of the flange 24 beyond the adjacent cylindrical surface is provided viewed in radial direction.

As evident from FIG. 9 the blocking tongues 79 are formed during injection molding of the main body parts 11, 12 so that they are inclined relative to the longitudinal axis 7 of the main body 2 or the sliding body 15 starting from a connection cross section with the associated main body part 11, 12 and moving towards their free ends. In order to maintain this preload radially inward reliably even during a long storage time of the safety cannula arrangement 1 relief grooves 45 are formed in the socket section 42 of the sliding body 15 as described supra. The relief grooves facilitate an orientation of the blocking tongues 79 that is inclined relative to the longitudinal axis 7 during storage in the operating condition of the sliding body exactly in the shape of the relief tongues that is subsequently required in the safety position of the sliding body 15 to block another deployment movement of the sliding body 15. This way material fatigue and a loss of the radially inward oriented preload is prevented compared to a configuration of the socket section 42 without the relief grooves 45 which could otherwise lead to a failure of the blocking tongues 79 so that a redeployment of the cannula 17 would not be prevented reliably.

The following is appreciated regarding the configuration of the trigger mechanism 37 with reference to FIG. 18, as well as FIGS. 10, 10A and 4.

As stated supra, the main body 2 is substantially cuboid in the trigger portion 36, wherein a respective trigger member 67a, 67b and the associated compression rod 66a, 66b in combination with link elements configured as bar 82 jointly form a L-shaped wall arrangement, wherein the three link elements are connected by the respective compression rod 66a, 66b. Due to the small cross sections of the bars 82, they can be deformed easily by pressure upon the trigger members 67a relative to the proximal closure portion 38. When pressure is imparted upon the trigger members 67a, 67b, a connection cross section 83 arranged between the trigger members 67a, 67b and the closure portion 38 functions as an additional pivot joint. A trigger surface respectively formed by the trigger member 67a, 67b runs at an angle of 90 degrees relative to a plane in which the bars 82 and the respective compression rod 66a, 66b connecting the bars 82 are arranged. The respective locking elements 61a, 61b extend at an angle of 90 degrees relative the plane of the bars 82, and the respectively associated compression rod 66a, 66b. Overall, a C- or U-arrangement is achieved in cross section.

### REFERENCE NUMERALS AND DESIGNATIONS

1 safety cannula arrangement
2 main body
3 lobe module
4 cannula protector
5 tube
6 transversal rib
7 longitudinal rib
8 longitudinal rib
9 grip portion
10 first main body part
11 second main body part
12 third main body part
13 film hinge
14 film hinge
15 sliding body
15m enveloping surface
16 distal end section
17 cannula
18 tip
19 proximal end section
20 distal end section
21 actuation element
21s support surface
22 distal end
23 proximal end
24 flange
25 interlocking lug
26 rotation symmetrical section
27 cuboid section
28 tip section
29 transition section
30 connection part
31 gripping lobe
32 length
33 length
34 overlapping portion
34d section
34p section
35 notched portion
36 trigger portion
37 trigger mechanism
38 closure portion
39 snap hook
40 connection bar
41 snap hook
42 socket section
43 transition section
44 plug in section
45 stress relief groove
46 section
47 section
48 boundary line
49 end section
50 diameter
51 diameter
52 center portion
53 recess
54 recess
55 base
56 semi-wall
57 semi-wall
58 face
59 face
60 free length
61a, 61b locking element
62 proximal face
63 shoulder
64a, 64b control edge
65 enveloping surface
66a, 66b compression rod
67a, 67b trigger member
68a, 68b protrusion
69a, 69b arrow
70 distance
71 circumferential line
72 inner cavity
73 stop surface
74 stop surface
75 arcuate line
76 stop element
77 section
78 stop surface
79 blocking tongue
80 window
81 face
82 bar
83 connection cross section
84 rotation blocking device

1. A safety cannula assembly, comprising:
a cannula configured to puncture human or animal tissue, wherein a distal end section of the cannula includes a tip;
a sliding body including a distal end section connected with the cannula and a proximal end section connected with a flexible tube, wherein a flow connection between the tip of the cannula and a proximal end of the flexible tube runs through the sliding body;
a main body including an interior space with the sliding body movably supported in the interior space;
an actuation element arranged between the main body and the sliding body and configured to displace the sliding body from an operating position where the tip of the cannula is located outside the main body to a safety position where the tip of the cannula is arranged inside the main body; and
a trigger mechanism arranged at the main body and configured to trigger a displacement of the sliding body from the operating position to the safety position, wherein the trigger mechanism includes at least one trigger member loadable with a compressive force by a finger of a person using the safety cannula assembly and provided with at least one locking element displaceable from a locking position where the locking element engages the sliding body and locks the sliding body in the operating position, to a trigger position, where the locking element does not engage the sliding body so that the sliding body moves into the safety position, wherein a distance of the at least one locking element from a longitudinal axis of the sliding body is increased when the at least one locking element is displaced from the locking position to the trigger position,
wherein the at least one trigger member and the associated at least one locking element are located on different or on opposite sides of the longitudinal axis of the sliding body.

2. The safety cannula assembly according to claim 1, wherein a respective trigger member of the at least one trigger member and a respective locking element of the at least one locking element are arranged at diametrically opposite sides of the longitudinal axis in a cross section orthogonal to the longitudinal axis of the sliding body.

3. The safety cannula assembly according to claim 2,
wherein the respective trigger member and an associated respective locking element jointly form a U-shape or C-shape in a cross section of the main body oriented orthogonal to the longitudinal axis of the sliding body, and
wherein the respective trigger member is arranged at a first arm of the U or the C and the associated respective locking element is arranged at a second arm of the U or the C.

4. The safety cannula assembly according to claim 3,
wherein each respective trigger member is connected with the main body by at least one link element, and
wherein the at least one link element is elastically and/or plastically deformable relative to the main body when the respective trigger member is displaced from the locking position into the trigger position.

5. The safety cannula assembly according to claim 4,
wherein the at least one link element is a bar, and
wherein plural link elements configured as bars running parallel to one another and spaced equidistant from one another are provided.

6. The safety cannula assembly according to claim 5, wherein the at least one link element is connected with a proximal end at a proximal end section of the main body and includes the at least one locking element at a distal end.

7. The safety cannula assembly according to claim 6,
wherein plural link elements advantageously configured as bars are connected at their distal ends by a compression rod running perpendicular to the bars,
wherein the compression rod connects a trigger member with the associated locking element.

8. The safety cannula assembly according to claim 7,
wherein the at least one trigger member includes a trigger surface that is arranged at an angle of 90 degrees relative to a plane in which the at least one link element is movably arranged and/or in which a compression rod connecting plural link elements is arranged, and
wherein the trigger surface further advantageously includes a hump shaped protrusion that is detectable by a person using the safety cannular arrangement.

9. The safety cannula assembly according to claim 8,
wherein the main body is substantially cuboid in a portion of the trigger mechanism, and
wherein two L-arrangements respectively including a trigger surface and a plurality of link elements jointly form a rectangle in a cross section orthogonal to the longitudinal axis of the main body.

10. The safety cannula assembly according to claim 9,
wherein the main body includes two main body parts which are arranged at least essentially mirror symmetrical to a plane running through the longitudinal axis of the main body in an assembled condition of its two main body parts, and
wherein the two main body parts are connected by snap hooks or interlocking lugs through positive form locking.

11. The safety cannula assembly according to claim 10, wherein the at least one locking element contacts a proximal face of the sliding body in the operating position.

12. The safety cannula assembly according to claim 11, wherein a proximal face of the sliding body is formed by a socket section into which a distal end of the tube is inserted.

13. The safety cannula assembly according to claim 12, wherein the at least one locking element contacts a shoulder of the sliding body in the safety position.

14. The safety cannula assembly according to claim 13, wherein the shoulder that is contacted by the at least one locking element in the safety position is formed by a flange radially protruding beyond a rotation symmetrical outer jacket of the sliding body.

15. The safety cannula assembly according to claim 14, wherein the flange includes a face at its distal end that forms a support surface for the actuation element configured as a coil spring.
